(19) United States
(12) Patent Application Publication
(10) Pub. No.: US 2008/0194639 A1
(43) Pub. Date: Aug. 14, 2008

(54) Proton Pump Inhibitors in the Treatment of Sleep Disturbance Due to Silent Gastroesophageal Reflux

(76) Inventors: Paula Fernstrom, Molndal (SE); Goran Hasselgren, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 11/912,954

(22) PCT Filed: May 3, 2006
(86) PCT No.: PCT/SE2006/000535

§ 371 Date: Oct. 29, 2007

Related U.S. Application Data
(60) Provisional application No. 60/680,932, filed on May 12, 2005.

(30) Foreign Application Priority Data
May 4, 2005 (SE) 0501041-8

(51) Int. Cl. A61K 31/4439; A61P 1/00

(52) U.S. Cl. 514/338

(57) ABSTRACT

The present invention relates to a new method of treatment of sleep disturbance due to silent gastro-esophageal reflux. In particular, the present invention relates to the use of certain proton pump inhibitors (PPIs) in said treatment.

### FIELD OF THE INVENTION

The present invention relates to a new method of treatment of sleep disturbance due to silent gastro-esophageal reflux. In particular, the present invention relates to the use of certain substituted benzimidazole compounds, possessing pharmacological activity as an inhibitor of H⁺,K⁺-ATPase, in said treatment.

### BACKGROUND AND DETAILED DISCLOSURE OF THE INVENTION

It is well established that recurrent symptomatic reflux episodes negatively influences sleep quality. Patients that have both upright and supine reflux (day and night time) have more extensive esophageal mucosal damage than the upright (daytime) refluxers. Reduced saliva production, decreased number of swallows and delayed esophageal clearance time during sleep contribute to the more extensive esophageal mucosal damage. Sleep is affected in about 50-60% of patients with gastroesophageal reflux disease (GERD). These patients may be functionally impaired during the day as a consequence of their symptoms/sleep disturbances.

It is known that infusion of acid even in non-GERD subjects causes arousals (Orr et al, Gastroenterology, 86, 814-819, 1984.) Patients could thus present to the physician with sleep disturbances but without symptoms of GERD. About 10-15% of the US population have cronic sleep disturbances and it is estimated that 50% of these may have underlying acid reflux that is disrupting their sleep. The sleep problems may erroneously lead to prescription of hypnotics. Since hypnotics suppress arousals a hypnotic could actually increase acid contact time since it has been shown that the acid clearance is progressively and extensively prolonged during deeper sleep levels (Orr, 1984). This may ultimately increase the risk of developing esophagitis (Johnson, DeMeester, American Journal of Digestive Diseases, 23(6), 498-509, 1978), ulceration of the esophagus, Barrett's esophagus and similar conditions.

The target patient population also includes those who have fragmented sleep with frequent arousals. Sleep causes partial amnesia, which implies that even if patients cannot recall having had a reflux episode, the reflux episode may still be the reason for disrupting/fragmenting the sleep.

A similar mechanism is seen in patients with reflux laryngitis or acid asthma among which, less than 50% actually experience heartburn.

The side effects with hypnotics are well established (i.e., addiction, decreased quality of life and productivity) and other treatments to address sleep disturbance are needed.

The present invention relates to sleep disturbance due to silent gastro-esophageal reflux, and this should be clearly differentiated from the nocturnal GERD/heartburn related sleep disturbance, since these patients are excluded.

The use of proton pump inhibitors for treatment of sleep disturbance caused by night time heartburn or regurgitation is previously described. These patients having GERD either present with endoscopic signs i.e., mucosal breaks in the esophageal mucosa or symptoms typical for GERD, such as heartburn or regurgitation. However. isolated sleep disturbance without any of the signs/findings above would not be linked to GERD by someone skilled in the art. The present invention relates to these patients and the newly established link between silent reflux of gastric contents in the esophagus causing, as the only symptom, sleep disturbance. These patients are not, according to present definitions of GERD, regarded as GERD patients. Thus, they instead have “silent gastro-esophageal reflux”, a separate entity and disease.

The present invention thus relates to the treatment of patients with sleep disturbance due to silent gastro-esophageal reflux by administering a therapeutically effective amount of certain substituted benzimidazole compounds known as proton pump inhibitors (PPI).

In order words the present invention relates to the treatment of sleep disturbance due to silent gastro-esophageal reflux, i.e. the patient does not experience heartburn symptoms or other typical or traditional reflux symptoms, e.g. regurgitation. The patient may awaken, or get a change in sleep level (arousals) in response to the reflux event.

We have surprisingly found that reflux of acidic contents into the esophagus causes arousals/awakening even if the reflux episode is not associated with heartburn, regurgitation or acid taste. This results in sleep disturbance with reduced sleep quality with reduced quality of life and productivity.

Consequently the present invention offers a unique feature by i) improving sleep ii) reduce the risk of developing esophagitis iii) prevent development of Barretts' esophagus/adeno carcinoma, and iv) ultimately reduce the use of hypnotics in this group of patients.

The present invention is the first to disclose the relation between endogenous acid secretion and sleep disturbance and/or arousels and to link in time link the arousels to the EEG, EOG, EMG, and/or EKG of the patient.

### The Active Ingredient

The first aspect of the present invention is the use of a proton pump inhibitor in the treatment of sleep disturbance due to silent gastro-esophageal reflux. The proton pump inhibitor can be any of omeprazole, esomeprazole, lansoprazole, rabeprazole, pantoprazole, pariprazole, tenatoprazole, ilaprazole and leminoprazole or a mixture thereof.

For the purpose of this application the term “proton pump inhibitor” (PPI) shall mean any substituted benzimidazole compound possessing pharmacological activity as an inhibitor of H⁺,K⁺-ATPase, including, but not limited to, omeprazole, lansoprazole, pantoprazole, rabeprazole, esomeprazole, pariprazole, tenatoprazole, ilaprazole and leminoprazole in neutral form or a salt form, a single enantiomer or isomer thereof, or other derivative like an alkaline salt of an enantiomer of the same.

Example of suitable salt form of PPI is an alkaline salt, such as Mg²⁺, Ca²⁺, Na⁺, K⁺ or Li⁺ salt.

Further a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. The above-listed compounds can also be used in their tautomeric form. Also included in the invention are derivatives of the compounds listed above which have the biological function of the compounds listed, such as prodrugs. An example of a suitable prodrug is

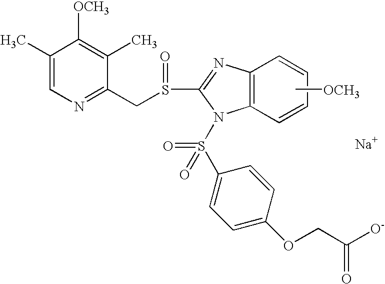

One aspect of the present invention is thus to administer to a subject suffering from sleep disturbance due to silent gastro-esophageal reflux a therapeutically effective amount of a proton pump inhibitor.

The invention relates in a further aspect to the use of PPIs for the treatment of patients who are suffering from sleep disturbance due to silent gastro-esophageal reflux.

The invention further relates to a method for the treatment of sleep disturbance due to silent gastro-esophageal reflux which consists in administering to a patient who needs such a treatment an effective amount of a PPI.

The invention further relates to the use of PPIs for the production of medicaments for the treatment of sleep disturbance due to silent gastro-esophageal reflux.

The invention further relates to a pharmaceutical preparation for the treatment of sleep disturbance due to silent gastro-esophageal reflux which contains at least one PPI as active compound.

In one embodiment of the present invention the pharmaceutical preparation is intended to give a immediate release profile.

In one embodiment of the present invention the pharmaceutical preparation is intended to give a modified release profile.

The invention further relates to a ready-to-use medicament, comprising a PPI as active compound, which contains a reference to the fact that this ready-to-use medicament can be employed for the treatment of sleep disturbance due to silent gastro-esophageal reflux.

The dosage form or forms, methods for preparing the pharmaceutical formulation and methods of administering the active ingredient and/or pharmaceutical formulation, including dosage levels and frequency,

### Immediate-Release Formulations

According to a one embodiment of the invention, there is provided an immediate-release pharmaceutical formulation comprising:

(a) an active ingredient (chosen from any of the compounds listed above), or a pharmaceutically-acceptable salt of any of these compounds; and
(b) a pharmaceutically-acceptable diluent or carrier,
which formulations are referred to hereinafter as “the immediate-release formulations of the invention” for the treatment of sleep disturbance due to silent gastro-esophageal reflux.

The term “immediate release” pharmaceutical formulation will be well understood by the skilled person to include any formulation in which the onset and/or rate of release, and/or absorption, of drug, is neither appreciably, nor intentionally, retarded by galenic manipulations. In the present case, immediate release may be provided for by way of an appropriate pharmaceutically-acceptable diluent or carrier, which diluent or carrier does not prolong, to an appreciable extent, the onset and/or rate of drug release/absorption. Thus, the term will be understood by those skilled in the art to exclude formulations which are adapted to provide for “modified” or “controlled” release, including a “sustained”, “prolonged”, “extended” or “delayed” release of drug.

In this context, the term “release” may be understood to include provision (or presentation) of drug from the formulation to the gastrointestinal tract, to body tissues and/or into systemic circulation.

In one embodiment of the present invention the immediate release formulation can include an aqueous solution/suspension of omeprazole or any other PPI, such as lansoprazole, in a pharmaceutically acceptable carrier including a bicarbonate salt of a Group IA metal. For the purposes of description, the composition includes both solutions and/or suspensions of the omeprazole or other substituted benzimidazoles. Suitable amounts of active ingredient and pharmaceutically acceptable diluent or carrier are as described in U.S. Pat. No. 6,489,346 which is incorporated by reference.

In accordance with the present invention, there is provided a pharmaceutical composition including an aqueous solution/suspension of omeprazole or any other PPI, such as lansoprazole, in a pharmaceutically acceptable carrier including a bicarbonate salt of a Group IA metal, preferably sodium bicarbonate, for the treatment of sleep disturbance due to silent gastro-esophageal reflux.

In one embodiment of the present invention, the immediate release formulation is Zegerid. Within the present invention is also an immediate release formulation substantially identical to Zegerid, but with another PPI as active ingredient instead of omeprazole.

### Modified Release

According to the invention there is provided a modified release pharmaceutical formulation comprising, an active ingredient, or a pharmaceutically-acceptable salt of an active ingredient, which formulations are referred to hereinafter as “the modified release formulations of the invention” for the treatment of sleep disturbance due to silent gastro-esophageal reflux.

The term “modified release” pharmaceutical formulation will be well understood by the skilled person to include any modified release formulation in which the onset and/or rate of release of drug is altered by galenic manipulations, and thus includes the definition provided in the United States Pharmacopeia (USP XXII) at pages xliii and xliv of the preface/preamble part, the relevant disclosure in which document is hereby incorporated by reference.

In the present case, modified release may be provided for by way of an appropriate pharmaceutically-acceptable carrier, and/or other means, which carrier or means (as appropriate) gives rise to an alteration of the onset and/or rate of release of active ingredient. Thus, the term will be understood by those skilled in the art to include modified release formulations which are adapted (for example as described herein) to provide for a “sustained”, a “prolonged” or an “extended” release of drug (in which drug is released at a sufficiently retarded rate to produce a therapeutic response over a required period of time, optionally including provision for an initial amount of drug being made available within a predetermined time following administration to cause an initial desired therapeutic response); modified release formulations which provide for a “delayed” release of drug (in which the release of drug is delayed until a specific region of the gastrointestinal tract is reached, following which drug release may be either pulsatile or further modified as indicated above); as well as so-called “repeat action” formulations (in which one dose of drug is released either immediately or some time after administration and further doses are released at a later time).

We prefer that the modified release formulations of the invention provide are substantially similar to those used in the commercially available PPIs (with the exception of Zegerid). Example of suitable commercially available PPIs are the following proprietary products; Nexium, Priolosec, Losec, Losec MUPS, Protonix, Prevacid, Aciphex, Omeprazole Eon, Omeprazole Impax Labs, Omeprazole Kremers Urban Dev, Omeprazole Lek Pharms, Omeprazole Mylan, Omeprazole Torpharm.

For the avoidance of doubt, by “treatment” we include the therapeutic treatment, as well as the prophylaxis, of a condition.

One aspect of the present invention is to administer a pharmaceutically active amount of the active ingredient at bedtime.

One aspect of the present invention is to administer a pharmaceutically active amount of the active ingredient twice daily.

One aspect of the present invention is to administer a pharmaceutically active amount of the active ingredient drug in a pulsatile mode.

Advantages with the present invention includes, but are not limited to, limiting the use of hypnotics to treat sleep disturbance, limiting the amount of fluid excreted by the stomach, reducing the intervariability between patients, more effective acid secretion inhibition than therapeutic amounts of other drugs with this effect.

### EXAMPLES

The following example is intended just as an Example and should not be seen as limiting the present invention.

### Example 1

Subjects meeting the entry criteria will undergo a history and physical exam. All subjects will undergo a standard drug screening test. Subjects will complete the Pittsburgh Sleep Quality Index, Functional Outcomes of Sleep Questionnaire, the Beck Depression Inventory, and the SF-36 for assessment of quality of life. All subjects will complete a daily sleep log for two weeks. At the end of this “run in” interval, an assessment will be made of the nights of disturbed or mornings with unrefreshed sleep. Subsequent to qualification, all subjects will undergo a full polysomnography (PSG) to include esophageal pH monitoring. All subjects will complete a questionnaire prior to bedtime and upon awakening in the morning. They are designed to assess activities of the current day and mental state prior to the sleep study, as well as morning mental state and subjective reports of awakenings and heartburn symptoms experienced during the sleep study.

The subject are then randomized into two groups, one that will get the active compound and one that will get placebo.

PSG Study:

The PSG study will consist of monitoring the EEG, EOG, EMG, and EKG. Respiration will be assessed via nasal oral sensor. The following parameters will be determined using standard internationally accepted criteria:
Total sleep time (TST)
Sleep onset latency (SOL)
Sleep efficiency (time asleep/time in bed)
Waking after sleep onset (WASO)
Arousal reponses (arousal responses will be defined by current AASM practice guidelines criteria).
Percent time REM sleep
Percent stage 3 and 4

### Esophageal pH Study:

A standard pH probe with dual sensors will be placed 5 cm above the manometrically determined proximal border of the lower esophageal sphincter (LES). The second pH sensor will be 5 cm proximal to the distal sensor. This will be accomplished at approximately 4:00 in the afternoon prior to each PSG study. The following pH parameters will be assessed:
Number of reflux events at the distal and proximal pH sensor
Arousal responses associated with reflux events (arousal responses will be defined as occurring within 5 minutes subsequent to the fall in pH below 4.0)
Average clearance time/event
Percentage acid contact time
Events exceeding 5 minutes duration

### Data Analysis

Data analysis will consist of comparing the two randomized groups, one on drug and one of placebo. The outcome will be compared in regard to the test discussed above, i.a. Pittsburgh Sleep Quality Index, Functional Outcomes of Sleep Questionnaire, the Beck Depression Inventory, the SF-36 for assessment of quality of life, polysomnography (PSG), Quality of Life in Reflux and Dyspepsia (QOLRAD) and esophageal pH.

### Example 2

104 patients with well documented sleep complaints, at least three nights a week were included. Patients were excluded if they had a history of GERD. The patients were randomized after a two week run in period with reorded sleep diaries. 81 patients completed two polysomnographic sleep evaluations including distal esophageal pH separated by 10-21 days. Of the 81 subjects studied, 26% had reflux (pH<4 for more than 30 sec) on at least one night. Of the partcipants with reflux, 21% had more than 4% acid contact time (ACT), 25% had at least one event that lasted more than five minutes and the average ACT was 28%. The average duration of each reflux episode was 34.4 minutes. Almost all (94%) of the recorded reflux events were associated with an arousal or awakening. An historical comparison of symptomatic GERD patients with concomitant sleep complaints had an average ACT of only 12% (p<0.05), see Orr et al, Am Journal of Gastroenterology 2005; 100 (Suppl 9):S50-51, Abs 82, enclosed herein by reference.

### Example 3

Sixteen subjects were enrolled into the study, eight potential reflux subjects and eight controls based on the Carlsson questionnaire. Patients were excluded if had a history of medications or exclusions such as a diagnosis of restless leg syndrome. periodic limb movement, Alzheimer's disease. Huntington's disease. Parkinson's disease, sleep apnea or a known history of gastro-oesophageal disease. The average Carlsson questionnaire score in the reflux group was 13.75 (range 10-17): the control group had a score of 0. There were 10 men and six women (age 22-62 years; mean 41.4 years). Body mass index was 25.7±1.2 (mean ± S.E.). Six subjects (75%) with a high Carlsson score and five subjects (62.5%) with a low score were found to have reflux events during sleep. The 11 subjects with reflux (six men and five women: age 22-62 years: mean 41.2 years) were evaluated. There were a total of 53 reflux events, which were associated with 41 awakenings and 128 arousals. All reflux events were associated with either an arousal or awakening or both. Subjects with reflux were analysed pre- and post-treatment with omeprazole. The number of awakenings (mean f S.E.) preceded by reflux events decreased from 3.7±0.9 to 1.3±0.5 (P<0.05). The number of arousals preceded by reflux events decreased from 11.6±3.8 to 1.5±0.8 (P<0.01). The total time (pH<4) decreased from 38.7±13.7 to 5.3±1.6 min (P<0.05). Six subjects with sleep efficiency<80% before omeprazole were further analysed. Data were analysed pre- and post-treatment with omeprazole. The total time (pH<4) decreased from 59.2±13.7 to 4.0±1.6 min (P<0.05) with omeprazole. Sleep efficiency improved from 70.2% to 81.6% (P<0.05). Total sleep time (mean ± S.E.) increased from 294.0+15.Y to 345.6+55.6 min (P<0.05), total awake time decreased from 99.1±17.9 to 46.1±15.3 min (P<0.05) and rapid eye movement sleep time improved from 55.0±4.5 to 94.5±18.9 min (P<0.05). In these six subjects with impaired sleep, number of awakenings decreased from 8.7±2.0 to 3.2±0.7 (P<0.01) and the number of arousals decreased from 3.3 f 1.2 to 1.5 k 0.6 (P<0.05), See DiMarina et al, Aliment Pharmacol Ther 2005: 22: 325-329, enclosed herein by reference.

Throughout this specification the following terminology apply, unless otherwise stated.

Reflux event—a decrease in the oesophageal pH below about 4.0.

Arousal—an abrupt frequency increase on EEG (except spindles) or wakefulness, lasting for at least about 3 s but less than about 15 s.

Awakening—a period of scored wakefulness lasting for at least about 15 s.

Sleep efficiency—the ratio of total sleep time to time spent in bed, expressed in percentage.

Sleep efficiency ˜80% was considered to be abnormal.

Total awake time—the cumulative amount of time spent awake during the recording.

Apnea—cessation of airflow at the nostrils and mouth for at least about 10 s.

1-3. (canceled)

4. A method for the treatment of sleep disturbance due to silent gastro-esophageal reflux, the method comprising administering an effective amount of a proton pump inhibitor (PPI) to a patient in need thereof.

5. The method according to claim 4, wherein the PPI is selected from the group consisting of omeprazole, lansoprazole, pantoprazole, rabeprazole, esomeprazole, tenatoprazole, ilaprazole and leminoprazole, wherein the PPI is in the form of the neutral compound, a pharmaceutically acceptable salt thereof a single enantiomer of the compound or a single enantiomer of the salt.

6. The method according to claim 4, wherein the PPI has the following structure: [chemical structure]
